(19) United States
(12) Patent Application Publication
(10) Pub. No.: US 2009/0306634 A1
(43) Pub. Date: Dec. 10, 2009

(54) Method and System for Control of Therapeutic Procedure

(75) Inventors: Jarbas Caiado de Castro Neto, Sao Paulo (BR); Mario Antonio Stefani, Sao Paulo (BR); Giuliano Rossi, Sao Paulo (BR); Alessandro Damiani Mota, Sao Paulo (BR); Rogerlo Alves Costa, Sao Paulo (BR); Jose Augusto Cardillo, Sao Paulo (BR); Jairo Kerr Azevedo, South Australia (AU)

Correspondence Address: JONES DAY, 222 EAST 41ST ST, NEW YORK, NY, 10017, US

(73) Assignee: Opto Global Pty Ltd, Thebarton, Adelaide (AU)

(21) Appl. No.: 11/990,301

(22) PCT Filed: Aug. 11, 2006
(86) PCT No.: PCT/AU2006/001147

§ 371 Date: Feb. 6, 2009

(30) Foreign Application Priority Data
Aug. 11, 2005 (AU) 2005904315

(51) Int. Cl. A61F 9/008; G06Q 50/00; G06Q 10/00; A61B 18/20

(52) U.S. Cl. 606/4; 705/2; 705/3

(57) ABSTRACT

A method and system are described for controlling a therapeutic procedure performed on a patient. Based on patient-related information, control system (90) determines at least one dosage parameter and at least one application parameter of the therapeutic procedure. Display (117) displays one or more prompts instructing an operator to introduce at least one external substance into the patient in accordance with the at least one dosage parameter and displays one or more instructions to the operator to apply an output of an application device (80) to a treatment area (100) of the patient in accordance with the at least one application parameter. The therapeutic procedure may be an Indocyanine Green mediated photothrombosis treatment for age-related macular degeneration.

### FIELD OF THE INVENTION

The present invention relates to medical treatment and diagnostic procedures. In a particular form the present invention relates to a control system and method for use in a therapeutic procedure that combines the introduction of external substances to a patient with the use of an application device.

### BACKGROUND OF THE INVENTION

A number of medical treatment and diagnostic procedures involve the combined effect of a substance which is introduced into a patient which in turn promotes the therapeutic or diagnostic effectiveness of a separate application device whose use forms part of the procedure. One example of such a procedure involves the introduction of a radioactive substance into a patient's body which is subsequently detected by an X-ray machine. The distribution of the radioactive substance throughout the areas being examined allows the clinician to determine the extent of conditions such as cancer and the like.

This procedure can also be applied in reverse where the introduced substance is a contrast or dye material which preferentially blocks X-ray photons as they pass through the body after emission from an X-ray machine. A similar principle applies in the use of contrast dyes and MRI machines where the application of the dye modifies the magnetic properties of the area being examined.

Another example of such a procedure is photodynamic therapy which involves the irradiation of certain chemicals which are selectively absorbed by cancer cells. On their breakdown under intense irradiation in the treatment area, these chemicals release further chemicals which are toxic to the cancer cells. Another procedure which relies on a similar principle is Indocyanine-Green Mediated Photothrombosis (i-MP) which is employed in the treatment of age-related macular degeneration (AMD) and other choroidal diseases.

AMD in its exudative stage is characterised by the formation of new blood vessels underneath the retina. This is termed choroidal neovascularisation (CNV) and these vessels tend to leak, causing haemorrhage and swelling of the macula leading potentially to retinal detachment, the formation of scars and ultimately to the irreversible loss of visual acuity. There are also other diseases that lead to the formation of CNV-type symptoms such as pathologic myopia, angioid streaks and other conditions resulting from idiopathic and inflammatory causes.

ICG-mediated photothrombosis (i-MP) is a procedure that relies on the photo-activation of Indocyanine Green (ICG) in the targeted tissue by the application of a continuous low irradiance 805 nm laser to achieve selective vascular occlusion with minimal or no damage to adjacent neural structures or tissues. The therapeutic effect arises from the photochemical reactions between pathologic tissues with increased ICG uptake and the laser energy causing selective necrosis of the CNV. The therapy may result in restoration or stabilisation of visual acuity and control of the disease.

However, treatment or diagnostic methodologies such as i-MP suffer from a number of significant issues which can make their use both overly complicated and costly. The most significant disadvantage is that these methodologies rely on the complex interplay between a chemical introduced into a patient and an application device such as a laser, X-ray machine or the like. Because of the complexity of the procedure there is greater scope for error either in the introduction of the relevant chemical to the patient and/or the use of what is often extremely sophisticated equipment in the course of the procedure.

### SUMMARY OF THE INVENTION

According to a first aspect of the invention there is provided a computer-implemented method of controlling a therapeutic procedure performed on a patient, the method comprising:
determining at least one dosage parameter and at least one application parameter of the therapeutic procedure dependent on patient-related data;
displaying one or more prompts instructing an operator to introduce at least one external substance into the patient in accordance with the at least one dosage parameter; and
presenting one or more instructions to the operator to apply an output of an application device to a treatment area of the patient in accordance with the at least one application parameter.

According to a further aspect of the invention there is provided a computer-implemented method of controlling a therapeutic procedure performed on a patient, the method comprising:
determining, dependent on patient-related data, a dosage of an external substance to be introduced into the patient;
calculating, dependent on the patient-related data, a desired output of an application device to be applied to a treatment area of the patient;
displaying prompts instructing an operator to introduce the external substance into the patient in accordance with a timing schedule of the therapeutic procedure; and
presenting instructions to the operator to apply the output of the application device to the treatment area, the instructions being presented according to the timing schedule.

According to a further aspect of the invention there is provided a computer-implemented method of controlling a procedure for treating macular degeneration in a patient's eye, the method comprising:
receiving data relating to the patient;
determining a quantity of an external substance to be introduced into the patient dependent on the received data;
calculating a desired power output of a laser to be applied to a treatment area in the patient's eye;
displaying prompts instructing an operator to introduce the external substance into the patient in a plurality of doses, wherein the prompts are displayed according to a timing schedule; and
presenting instructions to the operator to apply the laser beam to the treatment area in a plurality of applications, the instructions being presented according to the timing schedule.

According to a further aspect of the invention there is provided a system for controlling a therapeutic procedure performed on a patient, the system comprising:
means for determining at least one dosage parameter and at least one application parameter of the therapeutic procedure dependent on patient-related data;
means for displaying one or more prompts instructing an operator to introduce at least one external substance into the patient in accordance with the at least one dosage parameter, and
means for presenting one or more instructions to the operator to apply an output of an application device to a treatment area of the patient in accordance with the at least one application parameter.

According to a further aspect of the invention there is provided a system for controlling a therapeutic procedure performed on a patient, the system comprising:
data storage for storing patient-related information;
a display for displaying information to an operator, and
a processor in communication with the data storage and the display and arranged to:
determine at least one dosage parameter and at least one application parameter of the therapeutic procedure dependent on the patient-related data;
cause the display of one or more prompts instructing an operator to introduce at least one external substance into the patient in accordance with the at least one dosage parameter; and
cause the display of one or more instructions to the operator to apply an output of an application device to a treatment area of the patient in accordance with the at least one application parameter.

According to a further aspect of the invention there is provided a computer program product comprising machine-readable program code recorded on a machine-readable recording medium, for controlling the operation of a data processing apparatus on which the program code executes to perform a method of controlling a therapeutic procedure performed on a patient, the method comprising:
determining at least one dosage parameter and at least one application parameter of the therapeutic procedure dependent on patient-related data;
displaying one or more prompts instructing an operator to introduce at least one external substance into the patient in accordance with the at least one dosage parameter, and
presenting one or more instructions to the operator to apply an output of an application device to a treatment area of the patient in accordance with the at least one application parameter.

According to a further aspect of the invention there is provided a computer program product comprising machine-readable program code recorded on a machine-readable recording medium, for controlling the operation of a data processing apparatus on which the program code executes to perform a method of controlling a therapeutic procedure performed on a patient, the method comprising:
determining, dependent on patient-related data, a dosage of an external substance to be introduced into the patient;
calculating, dependent on the patient-related data, a desired output of an application device to be applied to a treatment area of the patient;
displaying prompts instructing an operator to introduce the external substance into the patient in accordance with a timing schedule of the therapeutic procedure; and
presenting instructions to the operator to apply the output of the application device to the treatment area, the instructions being presented according to the timing schedule.

According to a further aspect of the invention there is provided a computer program comprising machine-readable program code for controlling the operation of a data processing apparatus on which the program code executes to perform a method of controlling a therapeutic procedure performed on a patient, the method comprising:
determining at least one dosage parameter and at least one application parameter of the therapeutic procedure dependent on patient-related data;
displaying one or more prompts instructing an operator to introduce at least one external substance into the patient in accordance with the at least one dosage parameter; and
presenting one or more instructions to the operator to apply an output of an application device to a treatment area of the patient in accordance with the at least one application parameter.

### BRIEF DESCRIPTION OF THE DRAWINGS

An illustrative embodiment of the present invention will be discussed with reference to the accompanying drawings wherein:

FIG. 1A shows schematically a laser system that includes a laser unit and an optical delivery path for delivering laser energy to a patient's eye;

FIG. 1B shows a laser system having a detector positioned at the end of the optical delivery path and providing a feedback signal for calibrating the laser unit;

FIG. 1C is a schematic diagram of an application device including the laser unit of FIG. 1A having a control system, display and user inputs enabling operator interaction with the laser unit;

FIG. 1D is a schematic diagram showing more detail of the system of FIG. 1C;

FIG. 2 is a flowchart diagram of a mode selection process in the system of FIGS. 1A-1D used as an i-MP application device;

FIG. 3 is a flowchart diagram of steps performed in the AUTO-CALIBRATION mode;

FIG. 4 is a flowchart diagram of a first set of steps performed in SET PARAMETER mode;

FIG. 5 is a flowchart diagram of a second set of steps performed in SET PARAMETER mode;

FIG. 6 is a flowchart diagram of a third set of steps performed in SET PARAMETER mode;

FIG. 7 is a flowchart diagram of a first set of steps performed in USER PREFERENCES mode;

FIG. 8 is a flowchart diagram of a second set of steps performed in USER PREFERENCES mode;

FIG. 9 is a flowchart diagram of a first set of steps performed in TREATMENT mode;

FIG. 10 is a flowchart diagram of a second set of steps performed in TREATMENT mode;

FIG. 11 is a flowchart diagram of a third set of steps performed in TREATMENT mode;

FIG. 12 is a flowchart diagram of a fourth set of steps performed in TREATMENT mode; and

FIG. 13 is a flowchart providing an overview of the therapeutic procedure.

In the following description, like reference characters designate like or corresponding parts throughout the several views of the drawings.

### DESCRIPTION OF ILLUSTRATIVE EMBODIMENT

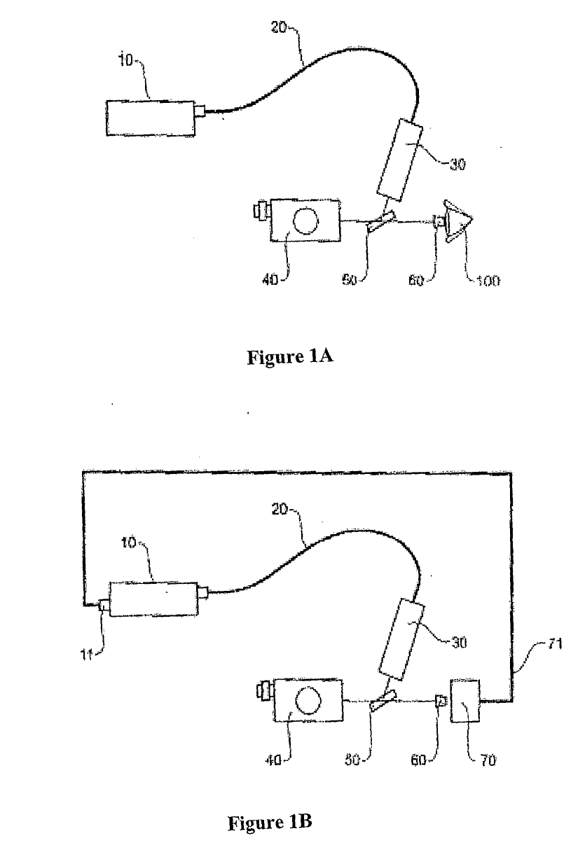

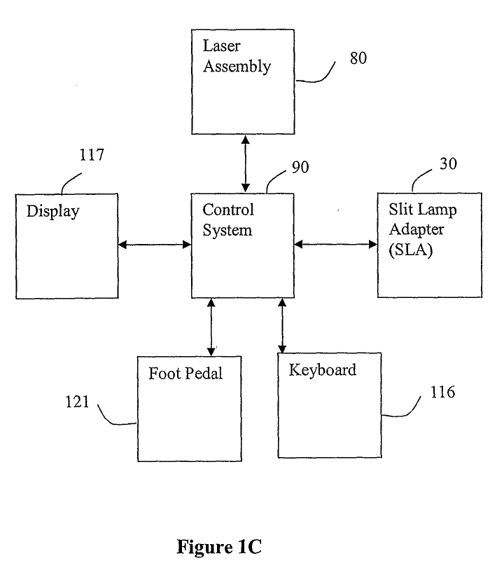

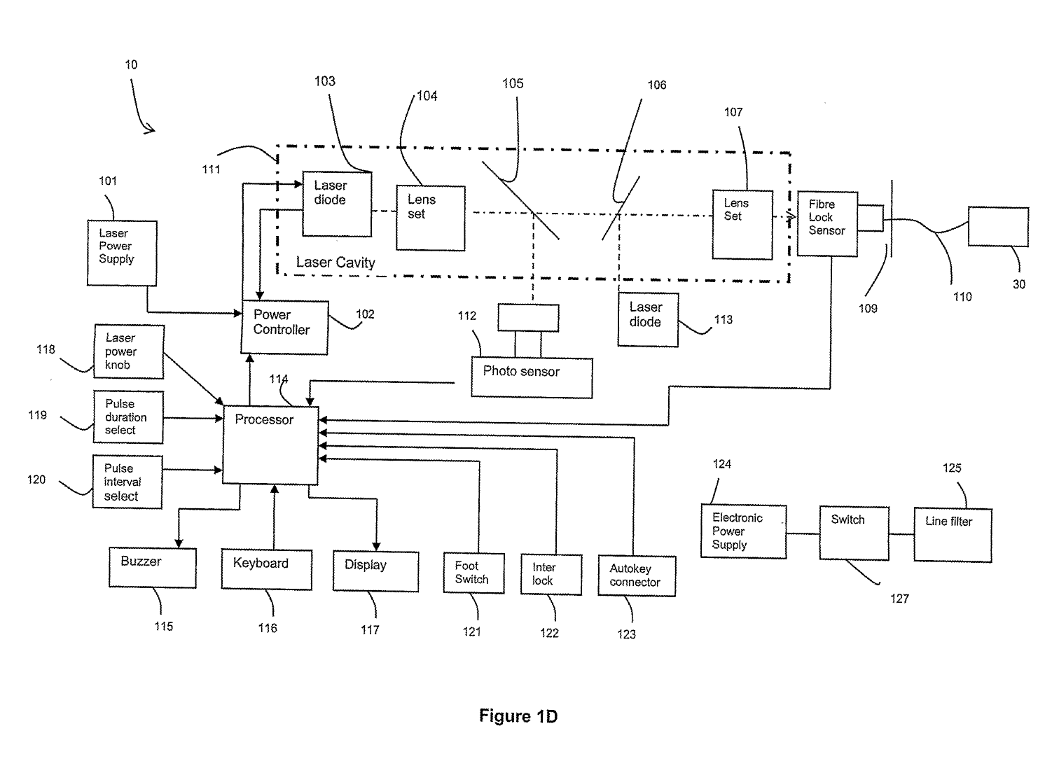

The laser system illustrated in FIG. 1A is an example of a photo-coagulator laser system and may be used in the application of a therapeutic procedure such as that described in WO 02/094260 “New use of Indocyanine Green as a Photosensitive Agent”, published on 28 Nov. 2002.

The photo-coagulator laser system includes a photo-coagulator laser unit 10 followed by an optical delivery path. Upon exiting the laser unit 10, the laser beam travels through the optical delivery path, which prepares and delivers the laser beam to a delivery point at a distal end of the optical delivery path. During treatment the delivery point is applied to the patient's eye 100. The optical delivery system generally includes fibre optic cable 20, slit lamp adaptor 30, slit lamp microscope 40, beam splitter 50, and a delivery end (contact lens 60). The contact lens 60 (during treatment) usually contacts the area of the eye that requires treatment, and allows the laser beam to pass through to the eye. Other types of optical delivery path may be used, including an endo-ocular probe, a laser indirect opthalmoscope and a surgical microscope adapter.

FIG. 1B shows an overview of a laser system incorporating an auto-calibration system. Detector 70 is placed behind the contact lens 60 so as to measure the power of the laser beam at the end of the delivery path. Detector 70 converts the measure of the power of the laser beam to an electrical signal which is then fed via communication link 71 to an input 11 of laser console 10. This electrical signal is converted into a digital signal (unless the signal is already a digital signal) which is then provided to a processor in laser console 10.

The measurement of the power of the beam made by the detector at the delivery end is then compared with the desired or required power level for delivery. This information is used to adjust a calibration factor associated with the optical delivery path. The calibration factor is used in controlling the power of the laser beam generated by laser console 10. Accordingly the power generation compensates for the effect of the optical delivery path.

This allows the power of the generated beam to be controlled to provide the desired laser power level to the patient, even though the optical delivery path may vary significantly for different procedures.

The auto-calibration also accounts for power deviations caused by component variation and degradation in the delivery path, as well as within the laser console itself.

The laser system calibration method is carried out at the practitioner's discretion, but preferably prior to use for each patient. In one arrangement the laser system locks to prevent more than ten procedures being performed without an auto-calibration. Once the laser system has been calibrated, the detector 70 is removed from the delivery point to allow treatment of the patient's eye 100.

Generally, deviations in transmission factors of the delivery system result in a loss of power of the laser beam, however if the laser system is calibrated to account for a loss and, for example, the laser system components are cleaned or replaced at a later stage, then the power of the laser delivered at the delivery end can become greater than that calibrated for, resulting in possible injury to the patient. Regular calibration avoids such problems.

Description of the Laser Console

Referring now to FIG. 1C, there is shown a system overview of the application device, laser unit 10, which may be employed in a therapeutic procedure according to an illustrative embodiment of the present invention. In this illustrative embodiment, the treatment or diagnostic system is the i-MP procedure described previously.

Whilst this illustrative embodiment is described with reference to the i-MP procedure the described arrangement may be applied to other medical procedures involving an application device used with externally introduced substances which when used together facilitate the medical procedure.

Application device 10 includes a control system 90, laser assembly 80, slit lamp adaptor (SLA) 30, display 117, keyboard 116 and foot pedal 121. Laser assembly 80 is a laser photocoagulator system which delivers controlled pulses of continuous wave 805 nm wavelength laser. The laser assembly 80 can deliver a maximum of 2.5 W of power which is continuously monitored by redundant safety systems. As illustrated in FIG. 1D, the laser console 10 includes a laser, a laser power supply, an electronics control board, an electronics power supply board, a control panel with display, keypad and buttons, a control panel board and a microcontroller board.

SLA 30 performs the function of delivering the laser beam to the patient's eye. It is an optical device including a fibre optic cable, a Galileo type microscope and a mechanical system which permits the device to be attached to a slit lamp microscope. SLA 30 is positioned coaxially with the optical path of the slit lamp microscope and allows the physician to apply the laser whilst viewing the back of the patient's eye (retina).

In one arrangement control system 90, keyboard 116, display 121 and laser assembly 80 are integrated into the same enclosure. Control system 90 is a microprocessor-based electronic circuit which runs the operational software and is responsible for controlling the operation of the laser assembly 80, aspects of the laser safety monitoring and interaction with the user interface including keyboard 116, display 117, user controls and foot pedal 121. Control system 90 also runs the routines which control the delivery of the treatment procedure. Control system 90 includes a microprocessor, memory, software, power supplies and other related electronics.

FIG. 1D shows the laser console 10 in greater detail and illustrates the system components included in the laser assembly 80 and control system 90. The main laser power supply 101 supplies the required current to produce the laser beam. The main laser power controller 102 is a module that controls the current to the main laser so that the output power is equivalent to the desired power. The laser diode 103 is used to generate the laser beam for the procedure. The wavelength of the laser is 805 nm, which is in the infrared range and is invisible to the human eye. The laser preferably has a tolerance of +/−3 nm. The laser produced by diode 103 passes through the main laser collimator lens set 104, which shapes the laser beam so that the beam can be focused onto the fibre-optic cable.

After the lens set 104, the beam passes through beam splitter 105, which is a partially reflective mirror that splits the laser beam, providing a percentage of the laser beam to a photo sensor 112 that forms part of a safety system.

The part of the beam that is not diverted by the beam splitter 105 reaches the aiming beam combiner 106, which is a special mirror that combines the main laser beam from diode 103 with an aiming laser beam received from laser diode 113. The aiming laser beam has a visible beam (red) that is used by the physician to aim the laser. In one arrangement the aiming beam laser has a wavelength of 630 nm and a maximum power of 1 mW. In contrast, the main beam has a maximum power of 2.5 W.

After the aiming beam combiner 106, the combined beam passes through a fibre coupler lens set 107 that focuses the laser beam onto the fibre optic cable of the optical delivery path.

Laser cavity 111 is a metal box which contains the main laser diode 103, and the optical components 104, 105, 106 and 107 used to adjust the shape, focus and direction of the laser. The aiming laser diode 113 may also be included in the laser cavity. The optical delivery path 110 is connected to an output nozzle of the laser cavity 111. The cavity 111 is sealed to protect the optical system from dust and humidity. At the output nozzle of the laser cavity 111, there is an optically-coupled fibre lock sensor 108 that indicates to the controller whether there is a fibre optic cable connected to the laser console 10. A mechanical laser shutter 109 is connected by a hinge to the laser console 10 to cover the output nozzle when no delivery device is connected to the laser console 10.

The laser console 10 may be connected to an optical delivery path 110 which includes a fibre optic cable used to deliver the laser beam to the patient's eye. Examples of optical delivery paths include an endo-ocular probe, a slit lamp adaptor, a laser indirect opthalmoscope and a surgical microscope adapter.

Some of the beam split by beam splitter 105 is provided to the main laser safety photo-sensor 112, which is a photodiode that reads the power level and provides an electronic signal used to ensure safe laser operation.

Processor 114 controls the functioning of all the laser equipment, and is in electronic communication with most of the components of the laser console 10. In one arrangement the processor 114 includes a microprocessor from the 8032 family, flash memory, e2prom and a watchdog unit. The processor 114 has access to data storage in which parameters of the therapeutic procedure may be stored. A buzzer 115 connected to the processor 114 is used to generate alarms, beeps and other audible signals.

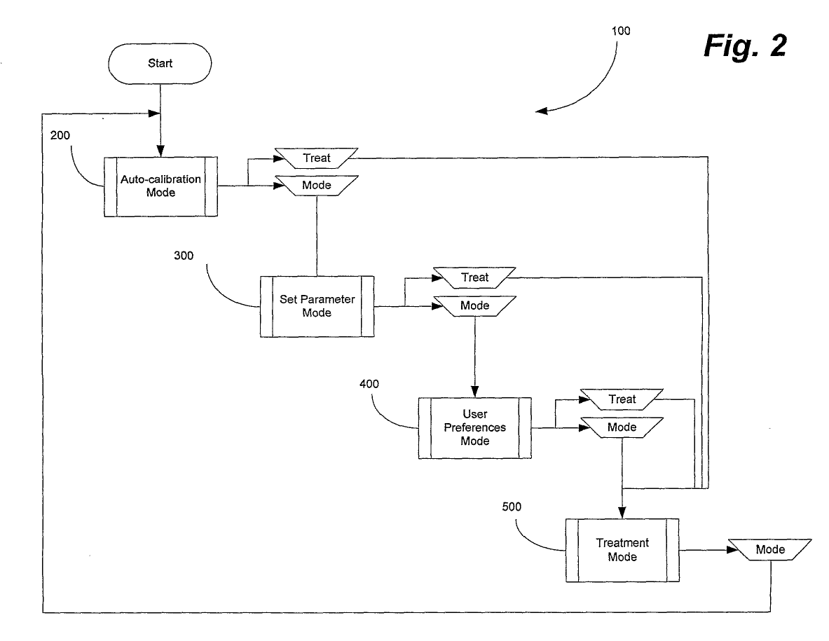

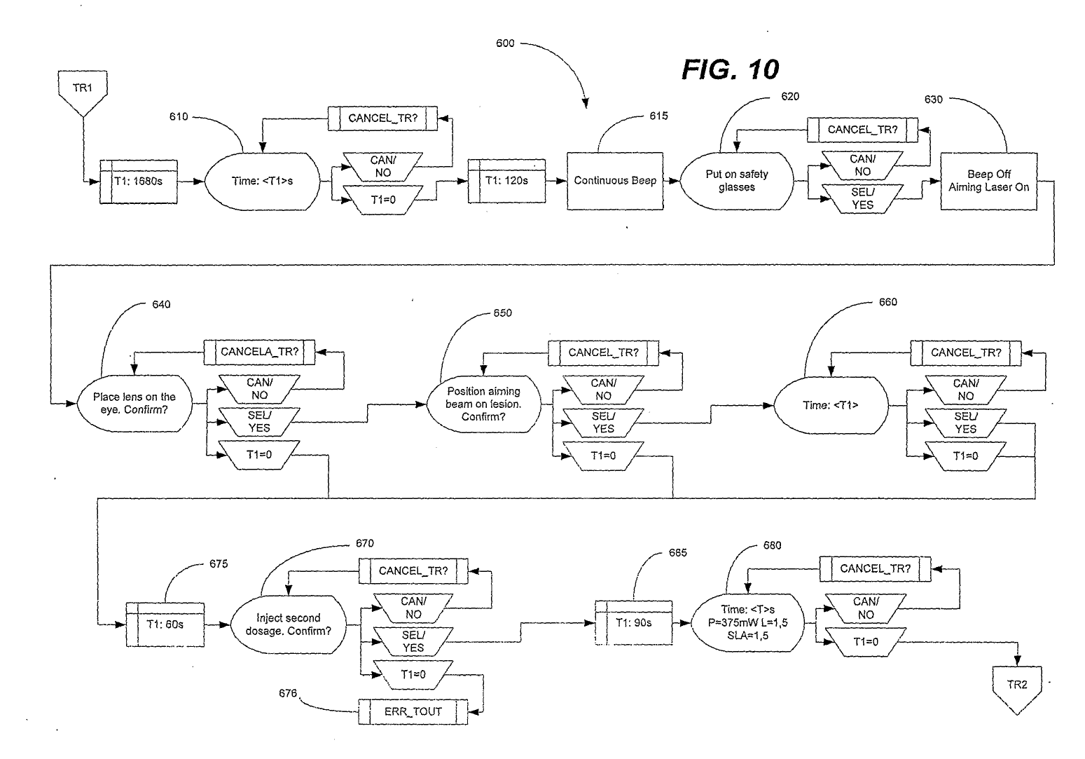

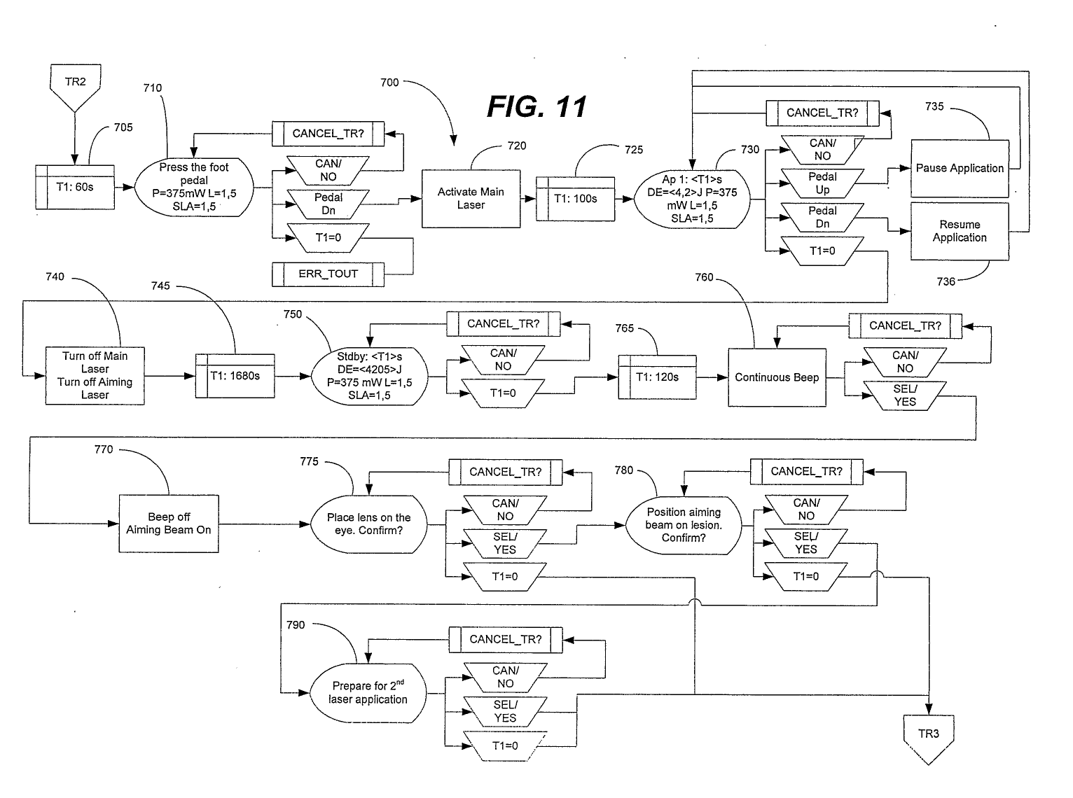

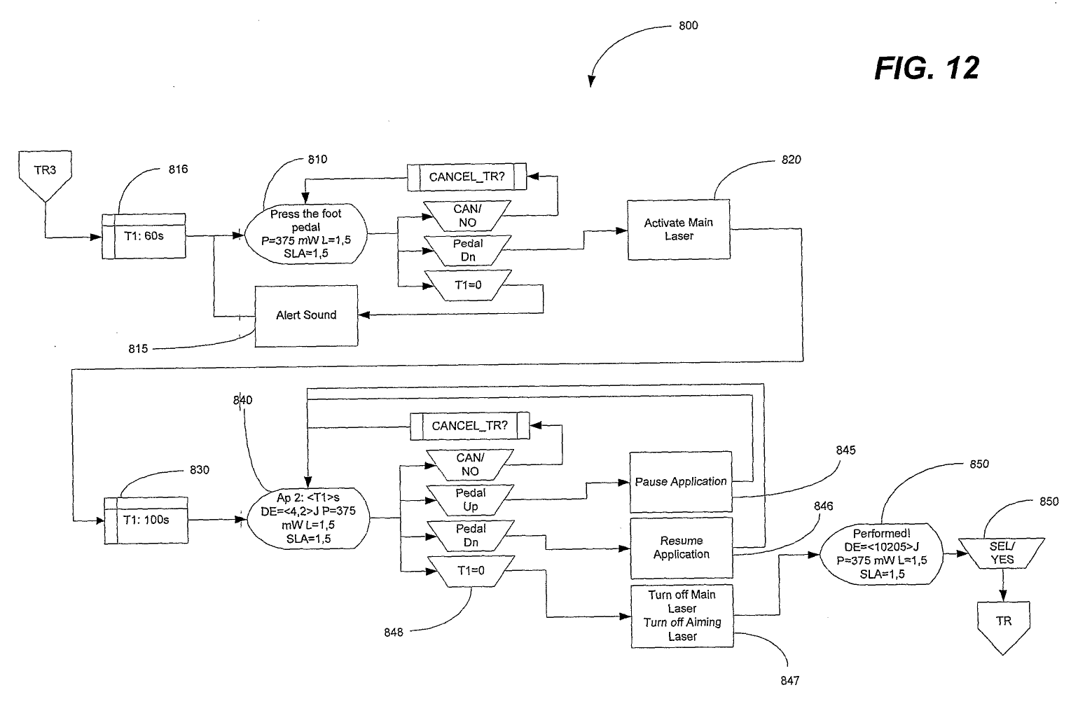

Keyboard 116 is used as an interface for the physician or operator to control the operating mode and parameters of the treatment, and the alphanumeric display 117 is used as an interface to show the treatment data and parameters to the physician using the laser console 10. As described in more detail with reference to FIGS. 2 to 13, the visual and audio outputs of the laser unit 10 may be used to guide an operator through the i-MP procedure.

A laser power knob 118 is preferably a rotary knob allowing the physician to set the main laser power. The power knob includes an encoder from which output signals are read and interpreted by the processor 114 and displayed to the physician.

The pulse-duration-select dial button 119 is a rotary knob allowing the physician to set the duration of a laser shot. The button 119 includes an encoder from which output signals are read and interpreted by the processor 114 and displayed to the physician, for example, on display 117.

The pulse interval select dial button 120 is a rotary knob which allows the physician to set the repeat interval. Diode button 120 includes an encoder from which output signals are read and interpreted by the processor board 114.

Foot switch 121 is used to fire the laser beam. The foot pedal 121 is optically coupled to the laser console 10 to provide electrical safety.

Interlock unit 122 is an optional device for additional laser safety. The interlock input 122 allows a switch to be connected to the laser console 10 to disable the laser when an external door is opened inadvertently. If the user chooses not to use the remote interlock, then a by-pass connector must be inserted into the interlock unit 122 to enable operation of the laser.

The “autokey” connector 123 contains electrical circuitry used to provide information to the laser console 10 that indicates what optical delivery path has been connected to the laser console 10. Each optical delivery path 110 has different transmission properties which affect the laser power that reaches the patient's eye 100. Information provided to the laser console 10 via the autokey connector 123 enables the console 10 to recognise the delivery device in use so that the processor 114 can calculate a transmission factor to compensate for the attenuation of laser power along the optical delivery path 110.

An electronic power supply 124 supplies the required power to the circuits of the power controller 102 and the processor board 114. EMI/EMC line filter 125 is a module that filters the electrical noise from the mains line to protect the laser from malfunction and damage due to possible power surges. Mains cable 126 connects the laser console 10 to an electric outlet. Switch 127 is an on/off switch allowing the user to turn the laser console 10 on or off.

Keyboard 116 includes a number of buttons for the operation of application device 10 and adjustment of the treatment parameters by an operator. The buttons include:
<Treat> Activates TREATMENT mode directly;
<MODE> Used to select the instrument's operating mode;
<SEL/YES> Selects or accepts the displayed option;
<INC> Increments the selected parameter;
<DEC> Decreases the selected parameters;
<CAN/NO> Cancels or declines the displayed option, and if pressed for some time, cancels the ongoing process;
<emergency> Emergency button—aborts all operations and places the device in emergency interruption mode.
<pedal> Foot pedal.

Selecting the Mode

Referring now to FIG. 2, there is shown a flowchart diagram of the mode selection process 100 of the treatment system. The treatment system has four modes of operation including:
AUTO-CALIBRATION mode 200: This mode is selected to calibrate the output power of laser unit 10. This calibration is necessary due to the precision required for the i-MP procedure. Auto-calibration is designed to compensate for any output power deviation arising either from accumulation of dust on the mirrors and lenses of the SLA 30, wearing out of the fibre optic, misalignment or aging of the laser diode 103. The adjustment range of the auto-calibration is 20% of the factory calibration thereby preventing the accidental use of the equipment out of the power tolerance specification.
SET PARAMETER mode 300: This mode includes a sequence of screens displayed on display 117 where the user is prompted to adjust the fundamental parameters of the treatment procedure including:
   Lesion greatest linear dimension (GLD);
   Patient's weight;
   Lens magnification; and
   Pigment concentration.
USER PREFERENCES mode 400: In this mode auxiliary parameters such as aiming beam intensity and sound intensity of buzzer 115 are adjusted by the operator.
TREATMENT mode 500: Mode in which the treatment laser 103 is applied to the patient using previously selected parameters.

Mode selection is accomplished by the operator pressing the <MODE> button repeatedly until the desired mode is displayed on display 117. Once a mode is shown on the display, pressing the <SEL/YES> button will commence the associated sequence of steps to be performed for that mode.

The procedures of FIGS. 2-12 are performed by software running on processor 114. Prompts are displayed to the operator on display 117 and the operator interacts with the software by pressing a button on keyboard 116 or pressing foot pedal 121. In some instances the operator is prompted to perform an action, for example putting on safety goggles or injecting the patient. The software procedure in general does not proceed until the operator has confirmed (by pressing a button on keyboard 116) that the action has been performed. For ease of description, the following text does not mention every point in the software where the operator is required to confirm that he or she wishes to proceed to the next step.

Auto-Calibration

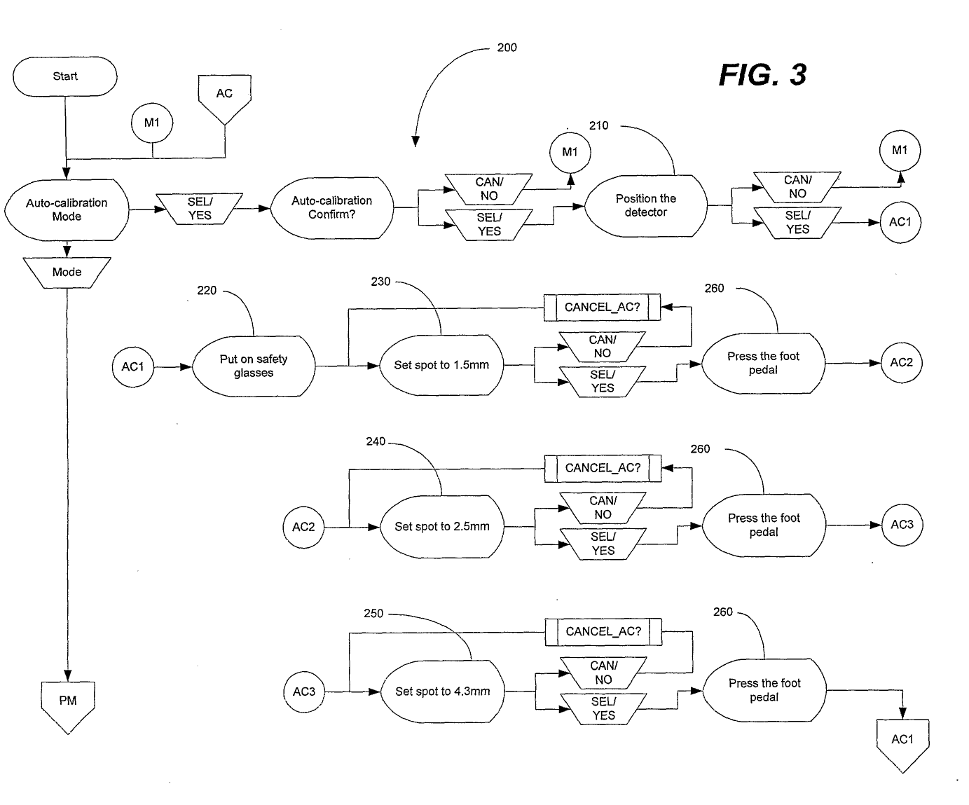

Referring now to FIG. 3, there is shown a flowchart diagram of the steps involved in guiding an operator through the auto-calibration of laser unit 10 with a particular optical path in place. AUTO-CALIBRATION mode 200 is used to fine tune the system's power control and compensate for any degradation and aging of the components. Dust on the mirrors, lenses and filters, micro-cracks in the fibre optical cable or misalignment of the fibre optic coupling are the most common causes of deviation of the output power of the laser. Additionally, the laser diode also ages and although this is somewhat compensated for by an internal closed-loop circuit there may be laser degradation to an extent that cannot be compensated by this circuit resulting in the requirement for external adjustment.

Application devices such as laser unit 10 are also governed by various standards which seek to ensure the safety of medical equipment. These standards stipulate that the power control must not exceed 20% of deviation. However, as the i-MP process is critically dependent on the irradiance of laser assembly 80, more accurate control down to the 5% level is required. The auto-calibration process involves the use of a purpose-designed power meter 70, which is placed in a position that corresponds to the location of the patient's eye 100 to measure laser power. The auto-calibration procedure is automatic, however the operator is required to position the power meter, connect the power meter cable 71 to the input 11 of the laser console 10 and activate the auto-calibration routine. As shown in FIG. 3, the laser console 10 prompts the user to perform the necessary actions.

To select AUTO-CALIBRATION mode 200 the operator presses <MODE> button on keyboard 116 repeatedly until display 117 shows the message:

<MODE> Auto-Calibration.

The operator then confirms that he or she wishes to complete the auto-calibration procedure and is then prompted 210 to position the power meter or detector 70 at which point the software running on processor 114 activates the aiming laser diode 113 to assist in the positioning.

After the operator has confirmed (by pressing the CAN/NO or SEL/YES buttons) that the detector 70 is positioned, the controlling software then prompts 220 the operator to wear his or her safety glasses before proceeding with the auto-calibration procedure. The first part of the procedure involves setting 230 a spot size to 1.5 mm. This is performed manually by the operator turning the thumb wheel on the SLA 30 to the 1.5 mm position at which point the operator is prompted 260 to either cancel the auto-calibration or press the foot pedal 121 thereby activating the laser diode 103 which will be fired for a period of time long enough to complete the internal calibration performed by software running on processor 114. The laser will be turned off and the user will then be prompted 240 to adjust the spot size to 2.5 mm and repeat the laser firing procedure. The software then prompts 250 the operator to adjust the spot size to 4.3 mm and once again activate the laser by pressing the foot pedal (prompt 260).

Depending on the results determined using the power measured by the detector 70, the operator will be informed of a successful calibration procedure or alternatively in the event of failure be prompted to take remedial action such as replacing the fibre 20 and/or cleaning the optics at which stage the auto-calibration procedure can be repeated.

The auto-calibration procedure is described in more detail in co-pending application PCT/AU2006/000721 “A laser calibration method and system” with an international filing date of 29 May 2006, the contents of which are incorporated herein by cross-reference.

Setting Parameters

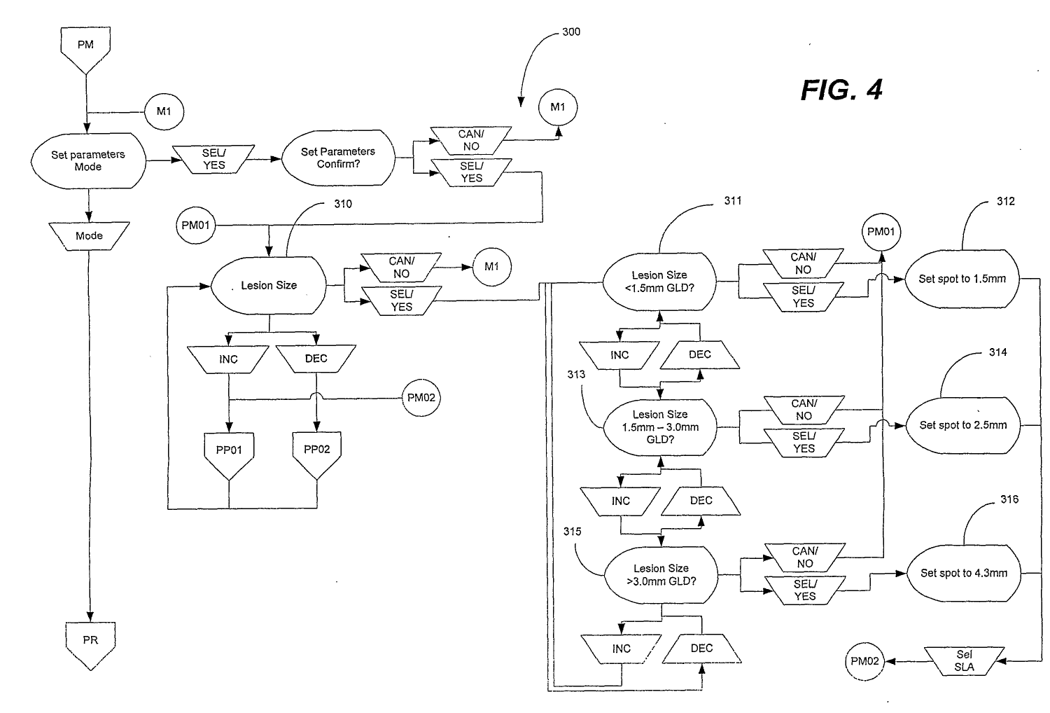

Referring now to FIGS. 4 to 6, there are shown flowchart diagrams of the steps involved in completing the setting of the necessary parameters required in the treatment procedure for the application device 10. The parameters include at least one dosage parameter, namely a quantity of ICG to be injected into the patient, and at least one application parameter such as the laser power to be used in the procedure. SET PARAMETER mode 300 guides the operator in entering the clinical parameters in order to determine the output power for the laser. The power setting of the laser is calculated by software running on processor 114 using the following equations:

P1=SZ*Mag*Kₗₐₛₑᵣ*K_{pig1}

P2=SZ*Mag*Kₗₐₛₑᵣ*K_{pig2}

where:

SZ=spot size selected at the SLA 30;

Mag=magnification of the retina laser lens 60 (typically 1.5);

Kₗₐₛₑᵣ=155.03875 W/mm² (Constant of Irradiance);

K_{pig1}=pigment factor 1;

K_{pig2}=pigment factor 2;

P1=output power in Watts for use in a first laser application; and

P2=output power in Watts for use in a second laser application.

Pigment factors 1 and 2 are based on an examination of the pigmentation of the patient's eye. In one arrangement the following values are used:

TABLE 1
Pigment factors

| Pigmentation | K_{pig1} | K_{pig2} |
|---|---|---|
| Low | 1.015 | 1.015 |
| Medium | 1.000 | 1.015 |
| High | 1.000 | 1.015 |

In the case of medium and high pigmentation, the power P2 used in the second laser application is higher than the power P1 used in the first laser application,

The output power is recalculated when the first and the second laser applications are started or when a fundamental parameter is changed. The output power is calculated by software running on processor 114.

To select SET PARAMETER mode 300 the operator presses the <MODE> button repeatedly until display 117 shows the message

<MODE> Parameter Adjustment

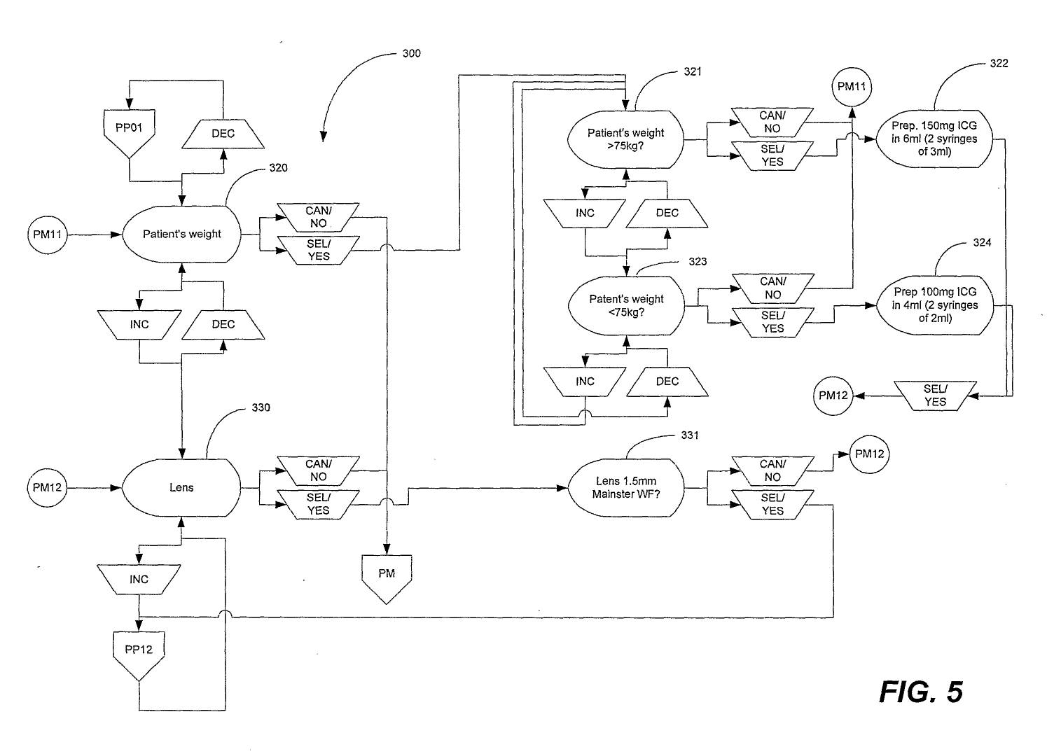

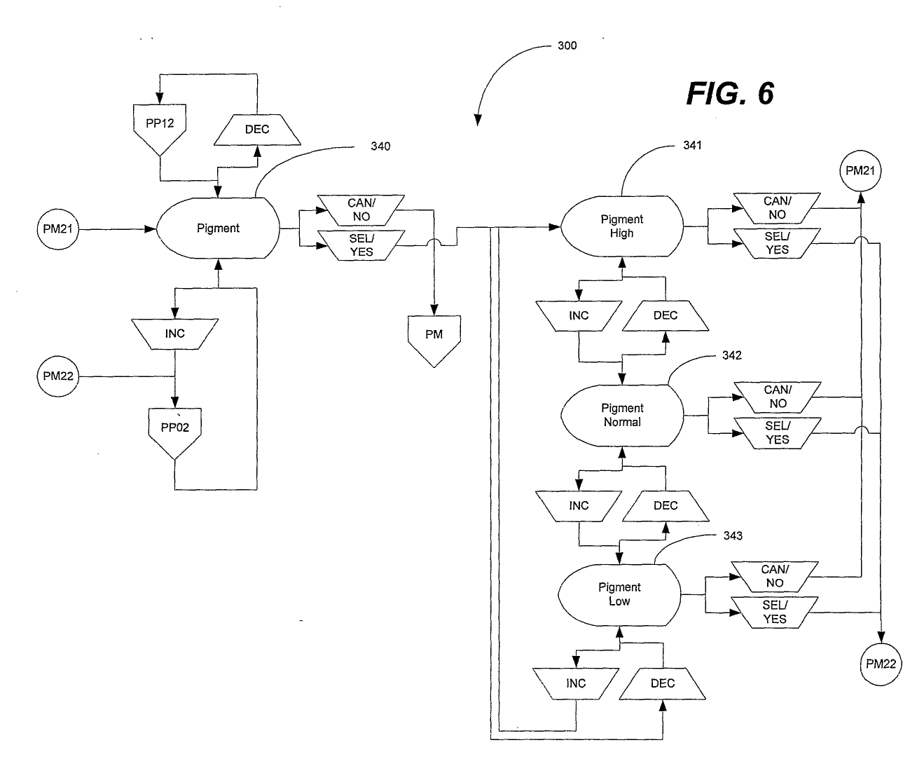

At this point the operator is presented with four selectable alternatives which include:
Lesion greatest linear dimension (GLD) 310 (see FIG. 4);
Patient's weight 320 (see FIG. 5);
Retina laser lens magnification 330 (see FIG. 5); and
Pigment type 340 (see FIG. 6).

The operator may step between these alternatives by pressing the INC and DEC buttons. The operator may then vary each of these options according to patient characteristics.

On selection of the lesion size mode 310 the operator is presented with three options to set the Lesion GLD. The choice between these options is based on an examination of the lesions in the patient's eye 100. The first of these options 311 corresponds to a lesion size less than 1.5 mm in which case the spot size (SZ) is to be set 312 to 1.5 mm. If this is appropriate as determined by examination of the patient, the operator is prompted 312 to turn the thumb wheel on SLA 30 to the appropriate spot size as indicated. The system will then return to mode selection level 200.

Alternatively, an operator can instruct the system to increase the lesion size parameter to be in the range 1.5 mm to 3.0 mm 313 in which case the spot size parameter stored in memory is set to 2.5 mm and the operator is prompted 314 to adjust the SLA 30 to a spot size of 2.5 mm. If the lesion size is indicated 315 as being greater than 3.0 mm, the spot size is set to 4.3 mm and a displayed message prompts 316 the operator to adjust the spot size at SLA 30.

Keyboard 116 allows the operator to move between the various options for lesion size by using the <INC> or <DEC> buttons as appropriate. Once the operator has selected the appropriate lesion size and confirmed this by pressing the SEL/YES button, the appropriate prompt 312, 314, 316 is displayed on display 117.

Another parameter of importance is the patient's weight as this will determine the amount of indocyanine green (ICG) that will be required to be injected into the patient. When prompt 320 is displayed, the operator has the choice of entering the patient's weight. After selection of patient's weight mode by pressing the SEL/YES button, the operator may enter whether the patient's weight is either under 75 kg 321 or over 75 kg 323 by pressing the INC or DEC buttons. If the patient's weight is over 75 kg then the operator is instructed (by prompt 322 displayed on display 117) to prepare 150 mg of ICG in two syringes of 3 ml each. Alternatively if the patient's weight is less than 75 kg then the operator is instructed (by prompt 324) to prepare 100 mg of ICG in two syringes of 2 ml each.

Once this has been completed, the operator returns to the SET PARAMETER menu to confirm 330 the type of contact lens 60 that is to be used. In the illustrated embodiment only a single lens option is provided. Prompt 331 asks the operator to confirm that a Mainster Wide Field 1.5 mm lens is used. In other embodiments, the operator may be presented with a potential choice of retinal laser lens types.

The final parameter to be selected in the SET PARAMETER mode is the pigment content of the eye that is to be treated. When prompt 340 is displayed on display 117, the operator is able to choose between a high pigment level 341, normal pigment level 342 and low pigment level 343 as determined by examination of the patient. The operator may step between prompts 341, 342 and 343 by pressing the INC and/or DEC buttons. When the appropriate pigment level is displayed on display 117, the operator selects the pigment level by pressing the SEL/YES button. Once this has been completed the process flow returns to the top level menu 200.

The parameters set in the SET PARAMETER mode are stored for use during the TREATMENT mode.

Setting User Preferences

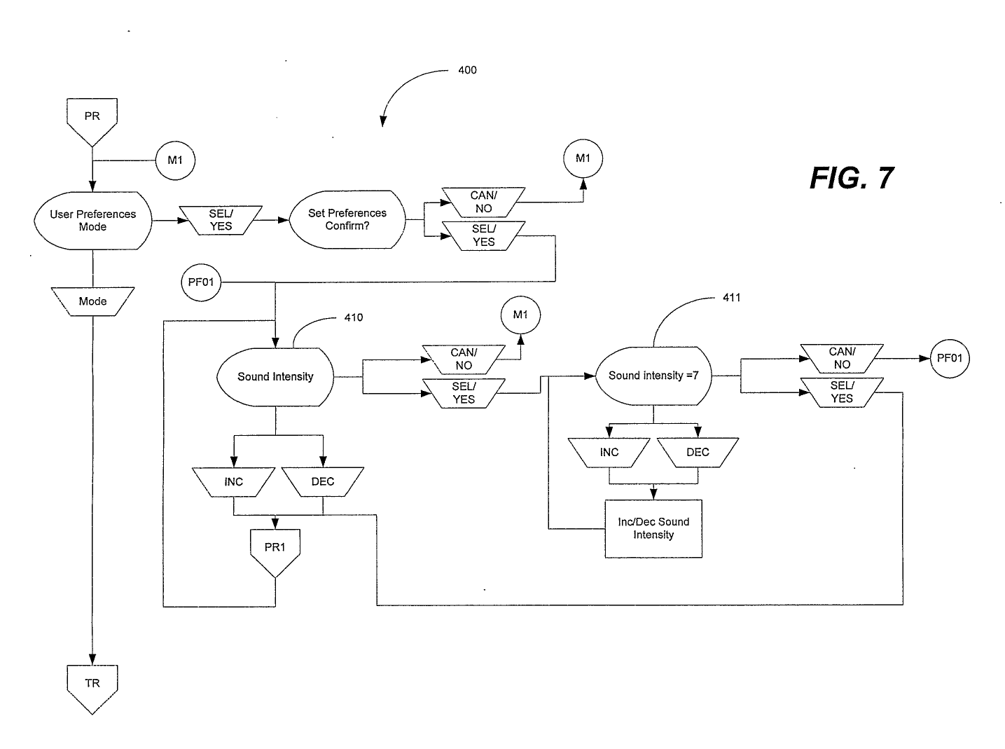

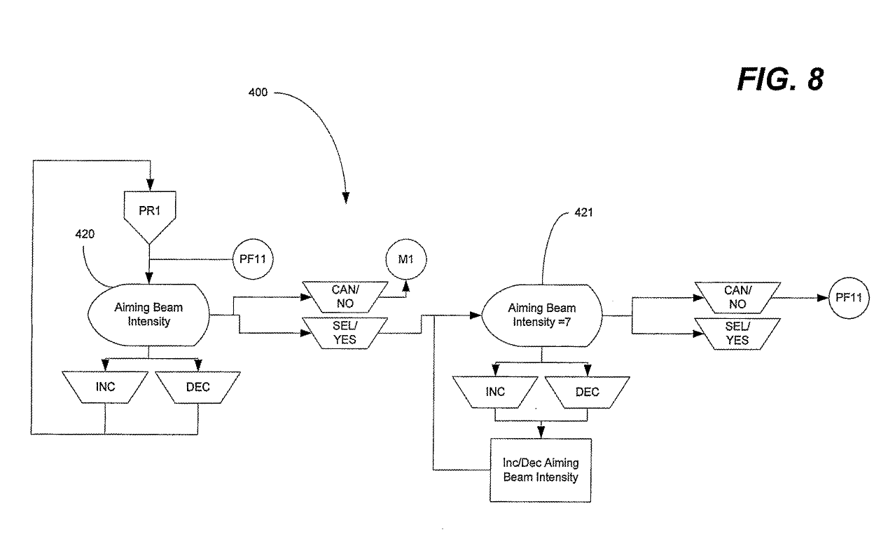

Referring now to FIGS. 7 and 8, there is shown a flowchart diagram of the steps involved in selecting features of the application device 10 which may be varied to suit the operator. In USER PREFERENCES mode 400, the operator can select some auxiliary parameters such as volume of the “beep” that is sounded by buzzer 115 and the intensity of the aiming laser beam emitted by laser diode 113.

To select USER PREFERENCES mode 400 the <MODE> button is pressed repeatedly until display 117 shows the message

<MODE> User Preferences

If the operator presses the MODE button again, the display changes to the next mode (TREATMENT MODE). Alternatively, if the operator enters the USER PREFERENCES mode by pressing the SEL/YES button, the operator is presented with a set of parameters to vary. The operator may step between these parameters by pressing the INC and/or DEC buttons on keyboard 116. Prompt 410 enables the operator to adjust the sound intensity level. By pressing the INC or DEC buttons, the sound intensity may be adjusted to a number ranging between 4 and 9. Displayed message 411 indicates the current setting of the sound intensity.

Similarly, as seen in FIG. 8, prompts 420 and 421 allow the operator to adjust the aiming beam intensity in a range of 2 to 9. As described above, the aiming beam is a visible laser of relatively low power output that is used to position and aim the laser subsequently used in the therapeutic procedure.

Treatment Mode

Once the various treatment parameters and user preferences have been adjusted according to the specific requirements of both the patient and the operator, the treatment protocol may be commenced.

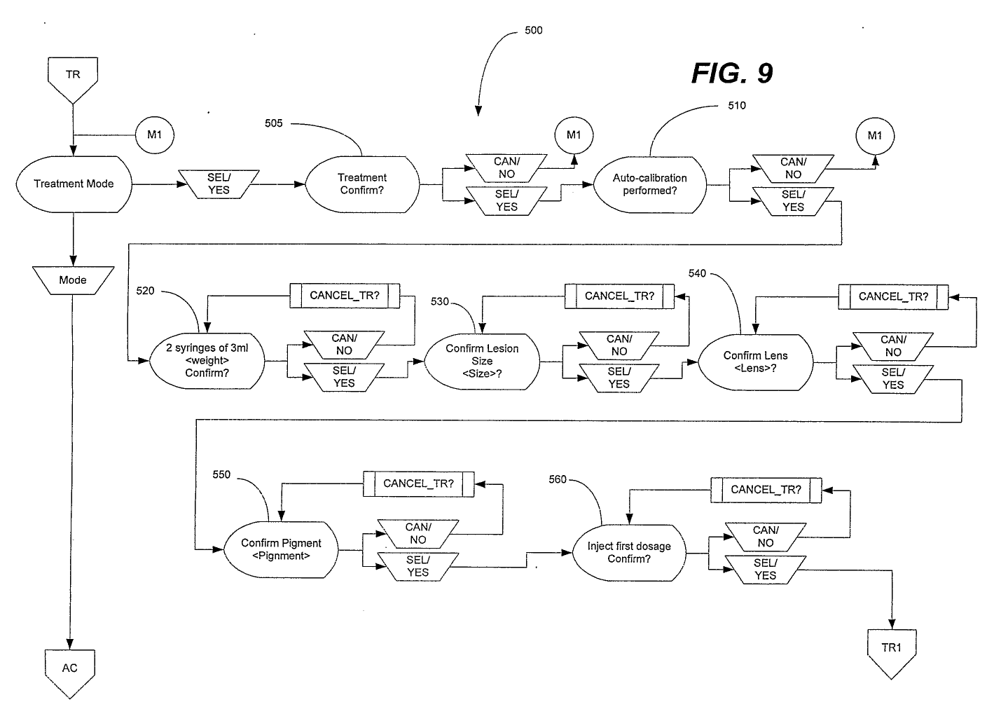

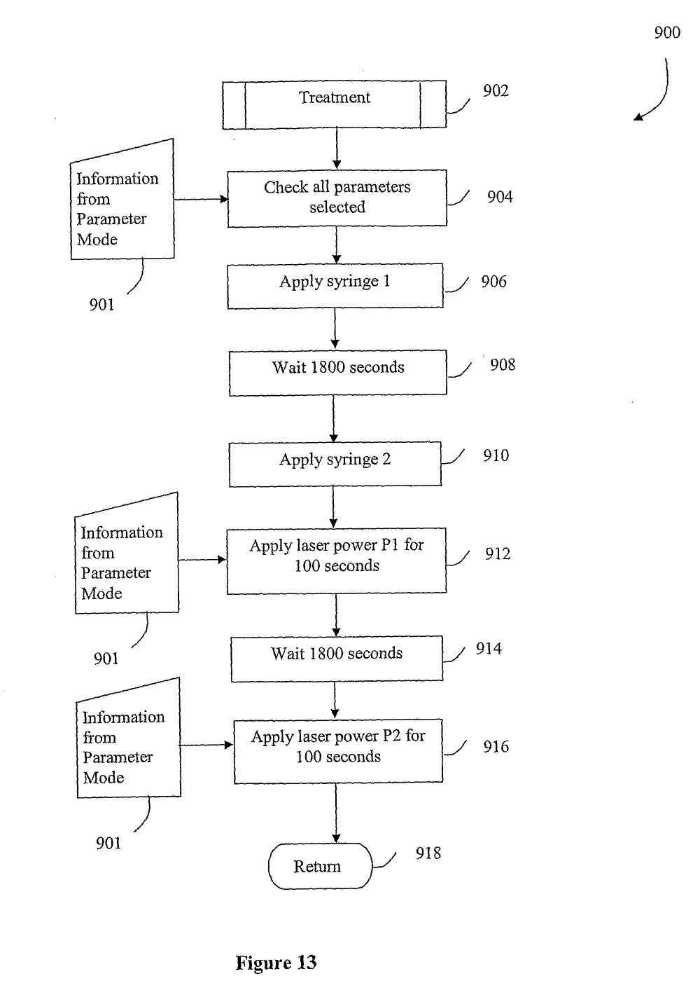

FIG. 13 provides an overview 900 of the treatment protocol. More detail is shown in FIGS. 9 to 12. The treatment protocol has a timing sequence. The control software described herein guides the operator in executing the treatment according to the timing sequence.

The procedure commences at 902. In step 904, information 901 saved during the SET PARAMETER mode is displayed on display 117 in order for the operator to check that the parameters have been selected appropriately. Next, in step 906, the operator injects a first syringe of ICG into the patient. After waiting 1800 seconds (step 908), the operator injects a second syringe of ICG into the patient (step 910). Software running on processor 114 guides the operator through steps 906-908 by providing prompts and instructions at appropriate times during the procedure.

After both syringes of ICG have been injected, in step 912 laser power P1 is applied to a treatment area in the patient's eye for 100 seconds. The operator applies the laser power by pressing foot pedal 121. The power setpoint of the laser is determined by software running on microprocessor 114 dependent on the information 901 entered in the SET PARAMETER mode.

After the first laser application there is a further wait of 1800 seconds (step 914). The control system 90 times the wait and alerts the operator near the end of the wait. Then, in step 916, laser power P2 is applied to the treatment area for 100 seconds. Information 901 entered in the SET PARAMETER mode is used to calculate the required laser power P2.

The procedure is then complete and the controlling software returns (step 918) to higher-level selection menus.

Referring now to FIGS. 9 to 12, there are shown flowchart diagrams of the steps 500 involved in guiding the operator through the ICG mediated photothrombosis procedure employing as application device the laser unit 10.

Once the operator has confirmed (by pressing the SEL/YES button in response to prompt 505) that a treatment run is to commence, the system initially prompts the operator to confirm that a number of important actions have been performed before the treatment can commence. In this manner, the system makes use of the various parameters that have already been entered into the system to ensure that the correct treatment protocol is being followed.

To select TREATMENT mode the <MODE> button is pressed repeatedly until display 117 shows the message

<MODE> Treatment

Alternatively, TREATMENT mode 500 may be activated directly by pressing the <Treat> button on keyboard 116.

In TREATMENT mode 500, the operator is first asked (by means of prompt 510 displayed on display 117) to confirm that an auto-calibration has been recently performed. In one arrangement the system requires that an auto-calibration is performed at least once every 10 treatments. Next the system confirms the weight of the patient and the amount of ICG that is to be delivered. For example, prompt 520 indicates that 2 syringes of 3 ml each are to be used, and asks the operator to confirm this. The operator responds by pressing the CAN/NO button or the SEL/YES button, as appropriate.

Following this, the selected lesion size is displayed 530 and the operator is prompted to confirm that the lesion size agrees with the spot size selected on SLA 30. The system then prompts 540 the operator to confirm which contact lens 60 is used. In the described example, a Mainster WF lens is used as the contact lens 60. The system also prompts 550 the operator to confirm that the pigment concentration parameter is correctly set. Prompts 510, 520, 530, 540, 550 are part of a final safety check to ensure that the system is parameterised correctly with regard to the patient to be treated.

The system next prompts 560 the operator to inject the first dose of ICG into the patient's blood circulation. After the first syringe has been injected, the operator confirms the injection by pressing the SEL/YES button. Alternatively, the operator may halt the procedure by pressing the CAN/NO button. If the injection is confirmed, software running on processor 114 sets a count down timer to 1680 seconds. The current value of the timer is displayed 610 on display 117. The operator may cancel the timer by pressing the CAN/NO button.

When the timer has finished the count-down, the controlling software resets the timer to 120 seconds and causes the buzzer 115 to sound a continuous beep (step 615). The system continues counting down and prompts 620 the operator to put on safety glasses and confirm that this has been done. If the operator presses the SEL/YES button to confirm use of the safety glasses, processor 114 switches off the buzzer 115 and switches on the aiming laser diode 113. A prompt 640 is then displayed instructing the operator to place lens 60 on the patient's eye and confirm that this has been done. Prompt 650 then instructs the operator to position the aiming laser beam on the lesion and to confirm that this action has been performed. The display 117 continues to display 660 the status of timer T1.

After the timer has completed the 120 second countdown (during which time the operator has put on safety glasses, positioned lens 60 and aimed the laser), the timer is reset to 60 seconds (step 675). Prompt 670 is displayed instructing the operator to inject the second syringe into the patient. The operator is required to confirm, by pressing the SEL/YES button, that the second syringe has been injected into the patient. If this confirmation has not occurred within 60 seconds the treatment will automatically time out and display 117 presents the message Treatment time-out 676.

The system then, in step 685, starts a 90 second countdown timer. Screen display 680 displays the timer and also the calculated power parameter P, the lens magnification and the spot size. In the example shown, P=375 mW, L=1.5 and the spot size is 1.5 mm. When the timer reaches the last 30 seconds of the countdown period, 3 long beeps are sounded. When the timer reaches the last 20 seconds, 2 long beeps are sounded and at the last 10 seconds one long beep will be emitted. At the last 5 seconds one beep is sounded at each second. At the end of the 90 second period, the system displays a prompt 710 instructing the user to press the foot pedal 121. If the operator does not press the foot pedal within 60 seconds (set in step 705), the procedure times out and an error signal is displayed. The operator also has the option of canceling the procedure by pressing the CAN/NO button.

If the operator presses the foot pedal 121, a sound signal is emitted by buzzer 115 and the main laser diode 103 is activated (step 720). At this stage, the patient is positioned and the lens is in place. A 100 second countdown timer is started in step 725. During the application of the main laser, the power, lens magnification and spot size are displayed 730 and an indicator of delivered energy dosage is also displayed. The display 730 also includes an indication that the first laser application is underway.

The foot pedal 121 must be kept pressed. If the foot pedal 121 is released the laser diode 103 and countdown are paused (step 735). The countdown and laser treatment can be resumed (step 736) by pressing foot pedal 121 again. In the event of foot pedal 121 not being pressed for more than 30 seconds, the system will display the time-out message. The operator may also cancel the procedure by pressing the CAN/NO button.

At the end of the 100 second period, the controlling software running on processor 114 deactivates the treatment laser diode 103 and aiming laser diode 113 (step 740). A 1680 second countdown timer is then started in step 745. The delivered energy (DE) dose indicator remains on display 750 whilst the laser assembly 80 is in standby mode. In the example shown the delivered energy is 4205 J.

When the 1680 second countdown is complete, the timer is reset to 120 seconds in step 765, providing an overall delay of 1800 seconds. The buzzer 115 sounds a continuous beep 760 to alert the operator. If the operator presses the SEL/YES button, the processor 114 acts (in step 770) to turn off the beep and turn on the aiming laser diode 113. The operator is then prompted 775 again to place the lens 60 on the eye 100 to be treated and to confirm that the lens 60 is in place. The software displays prompt 780 to instruct the operator once again to position the aiming laser on the treatment area including the lesion. If the operator confirms that this has been done (by pressing the SEL/YES button), then prompt 790 is displayed, prompting the operator to prepare for the second laser application. If the operator presses the SEL/YES button or the countdown timer reaches the end of the 120 seconds, the timer is reset to 60 seconds in step 816 and prompt 810 is displayed instructing the operator to press the foot pedal. The settings of power P, lens magnification and spot size are also displayed.

If the operator does not press the foot pedal within the 60 second window, then in step 815 the buzzer 115 sounds an alert.

Once the foot pedal 121 is pressed the main laser diode 103 is activated in step 820 and a countdown period of 100 seconds commences (step 830). An indicator of energy dosage is displayed 840 along with power, lens magnification and spot size and an indication that this is the second application of the laser assembly 80. As described above, the power P2 used in the second laser application may be higher than the power P1 used in the previous laser application. The relative strength of P1 and P2 is determined by the pigmentation parameter K_{pig}.

The foot pedal 121 must be kept pressed down. If the foot pedal 121 is released the laser and countdown are paused (step 845). These can be resumed (step 846) by pressing the foot pedal 121 again. If the foot pedal 121 is not pressed for more than 30 seconds, application device 10 will sound a continuous beep until the foot pedal 121 is pressed again.

At the end of the 100 second period, the treatment laser diode 103 and aiming laser diode 113 are deactivated in step 847. The delivered energy dose indicator remains on the display 117 (step 850) and the operator is given the option to return to the top level menu display 200. At various stages throughout the procedure the treatment may be cancelled explicitly by the operator or alternatively if a time out period has been exceeded.

As would be apparent to those skilled in the art the treatment or diagnostic procedures that are contemplated to be within the scope of the present invention may equally be applicable to humans or animals.

Although an illustrative embodiment of the present invention has been described in the foregoing detailed description, it will be understood that the invention is not limited to the embodiment disclosed, but is capable of numerous rearrangements, modifications and substitutions without departing from the scope of the invention as set forth and defined by the following claims. For example, WO 02/094260, the contents of which are incorporated herein by cross-reference, describes a range of laser wavelengths from 700 to 900 nm (preferably 805 nm) and an exposure time from 40 to 150 seconds (preferably 100 seconds). The dosage of ICG used may also vary, and different lenses may be placed in the path of the laser beam.

The control system described above is implemented by software running on a processor preferably included within the laser unit 10. However, some or all of the software-implemented tasks such as calculating the treatment parameters and displaying instructions to the operator may be run on other computational devices, which may be in electronic communication with one another and/or the laser unit. The software instructions may be stored on a computer-readable medium, for example floppy disks, magnetic tape, CD-ROM, DVD, a hard disk drive, a magneto-optic disk, or a computer-readable card such as a PCMCIA card and the like. A computer-readable medium having software or a computer program recorded on it is a computer program product. One or more aspects of the described system may also be performed in dedicated hardware, for example an application-specific integrated circuit.

It is apparent from the above that the described arrangements are applicable to the computer and data processing industries and to the field of medical treatment.

1. A computer-implemented method of controlling a therapeutic procedure performed on a patient, the method comprising: determining at least one dosage parameter and at least one application parameter of the therapeutic procedure dependent on patient-related data; displaying one or more prompts instructing an operator to introduce at least one external substance into the patient in accordance with the at least one dosage parameter; and presenting one or more instructions to the operator to apply an output of an application device to a treatment area of the patient in accordance with the at least one application parameter.

2. A method according to claim 1 wherein the therapeutic procedure has a timing sequence and wherein said displaying and presenting steps display prompts and present instructions according to the timing sequence.

3. A method according to claim 2 further comprising interrupting the therapeutic procedure if at least one specified action is not completed within a specified time.

4. A method according to claim 1 further comprising: requesting the operator to enter the patient-related data.

5. A method according to claim 1 further comprising: adjusting at least one setting of the application device dependent on the at least one application parameter, the output of the application device being dependent on the at least one setting.

6. A method according to claim 1 further comprising: prompting the operator to operate the application device according to a predetermined calibration procedure to calibrate the application device for the therapeutic procedure.

7. A method according to claim 1 further comprising: displaying safety information related to the therapeutic procedure, the safety information being displayed at one or more predetermined stages of the therapeutic procedure.

8. A method according to claim 1 wherein the therapeutic procedure is a procedure for treating age-related macular degeneration.

9. A method according to claim 8 wherein the external substance is indocyanine green (ICG) that is introduced into the patient by injection.

10. A method according to claim 1 wherein the application device is a laser.

11. A method according to claim 1 wherein the patient-related data comprises at least one of: a weight of the patient; a maximum dimension of a lesion in an eye of the patient; and a level of pigmentation in the eye of the patient.

12. A method according to claim 1 wherein the dosage parameter is a quantity of the external substance to be introduced into the patient.

13. A method according to claim 5 wherein the application device is a laser and wherein said adjusting step adjusts a power output of the laser.

14. A computer-implemented method of controlling a therapeutic procedure performed on a patient, the method comprising: determining, dependent on patient-related data, a dosage of an external substance to be introduced into the patient; calculating, dependent on the patient-related data, a desired output of an application device to be applied to a treatment area of the patient; displaying prompts instructing an operator to introduce the external substance into the patient in accordance with a timing schedule of the therapeutic procedure; and presenting instructions to the operator to apply the output of the application device to the treatment area, the instructions being presented according to the timing schedule.

15. A computer-implemented method of controlling a procedure for treating macular degeneration in a patient's eye, the method comprising: receiving data relating to the patient; determining a quantity of an external substance to be introduced into the patient dependent on the received data; calculating a desired power output of a laser to be applied to a treatment area in the patient's eye; displaying prompts instructing an operator to introduce the external substance into the patient in a plurality of doses, wherein the prompts are displayed according to a timing schedule; and presenting instructions to the operator to apply the laser beam to the treatment area in a plurality of applications, the instructions being presented according to the timing schedule.

16. A system for controlling a therapeutic procedure performed on a patient, the system comprising: means for determining at least one dosage parameter and at least one application parameter of the therapeutic procedure dependent on patient-related data; means for displaying one or more prompts instructing an operator to introduce at least one external substance into the patient in accordance with the at least one dosage parameter; and means for presenting one or more instructions to the operator to apply an output of an application device to a treatment area of the patient in accordance with the at least one application parameter.

17. A system according to claim 16 wherein the therapeutic procedure has a timing sequence, the system comprising: timing means for causing the display of the prompts and the presentation of the instructions according to the timing sequence.

18. A system according to claim 16 further comprising the application device.

19. A system according to claim 16 further comprising: means for determining one or more calibration factors for the application device.

20. A system for controlling a therapeutic procedure performed on a patient, the system comprising: data storage for storing patient-related information; a display for displaying information to an operator; and a processor in communication with the data storage and the display and arranged to: determine at least one dosage parameter and at least one application parameter of the therapeutic procedure dependent on the patient-related data; cause the display of one or more prompts instructing an operator to introduce at least one external substance into the patient in accordance with the at least one dosage parameter; and cause the display of one or more instructions to the operator to apply an output of an application device to a treatment area of the patient in accordance with the at least one application parameter.

21. A system according to claim 20 wherein the therapeutic procedure has a timing sequence and wherein said processor is arranged to cause the display of the prompts and instructions according to the timing sequence.

22. A system according to claim 20 further comprising: an audio output to provide audible signals relating to the timing sequence.

23. A system according to claim 20 further comprising the application device.

24. A system according to claim 23 further comprising a meter to measure the output of the application device, wherein the processor is arranged to calculate at least one calibration factor dependent on the measured output.

25. A system according to claim 20 wherein the application device is a laser.

26. A computer program product comprising machine-readable program code recorded on a machine-readable recording medium, for controlling the operation of a data processing apparatus on which the program code executes to perform a method of controlling a therapeutic procedure performed on a patient, the method comprising: determining at least one dosage parameter and at least one application parameter of the therapeutic procedure dependent on patient-related data; displaying one or more prompts instructing an operator to introduce at least one external substance into the patient in accordance with the at least one dosage parameter; and presenting one or more instructions to the operator to apply an output of an application device to a treatment area of the patient in accordance with the at least one application parameter.

27. A computer program product comprising machine-readable program code recorded on a machine-readable recording medium, for controlling the operation of a data processing apparatus on which the program code executes to perform a method of controlling a therapeutic procedure performed on a patient, the method comprising: determining, dependent on patient-related data, a dosage of an external substance to be introduced into the patient; calculating, dependent on the patient-related data, a desired output of an application device to be applied to a treatment area of the patient; displaying prompts instructing an operator to introduce the external substance into the patient in accordance with a timing schedule of the therapeutic procedure; and presenting instructions to the operator to apply the output of the application device to the treatment area, the instructions being presented according to the timing schedule.

28. A computer program comprising machine-readable program code for controlling the operation of a data processing apparatus on which the program code executes to perform a method of controlling a therapeutic procedure performed on a patient, the method comprising: determining at least one dosage parameter and at least one application parameter of the therapeutic procedure dependent on patient-related data; displaying one or more prompts instructing an operator to introduce at least one external substance into the patient in accordance with the at least one dosage parameter; and presenting one or more instructions to the operator to apply an output of an application device to a treatment area of the patient in accordance with the at least one application parameter.

29-31. (canceled)
